(19) United States
(12) Patent Application Publication
(10) Pub. No.: US 2025/0082945 A1
(43) Pub. Date: Mar. 13, 2025

(54) COILED RING CONTACTS FOR ELECTRICAL STIMULATION SYSTEMS AND METHODS OF MAKING AND USING SAME

(71) Applicant: Boston Scientific Neuromodulation Corporation, Valencia, CA (US)

(72) Inventors: Ranjan Krishna Mukhari Nageri, Valencia, CA (US); Austin Cisna, Saint Paul, MN (US); Alexander Daniel Gredzens, Delano, MN (US); Himanshu Ranpura, Stevenson Ranch, CA (US)

(21) Appl. No.: 18/882,583

(22) Filed: Sep. 11, 2024

Related U.S. Application Data
(60) Provisional application No. 63/538,241, filed on Sep. 13, 2023.

(51) Int. Cl. A61N 1/375

(52) U.S. Cl. CPC A61N 1/3754

(57) ABSTRACT

A connector assembly includes a coiled ring contact having a contact housing with a first part and a second part attached to the first part, wherein at least the first part includes a first radial sidewall and a first lateral sidewall extending laterally from the first radial sidewall, the first radial sidewall and the first lateral sidewall defining a pocket. The coiled ring contact also includes a coiled ring disposed within the contact housing, wherein the coiled ring contact is configured for disposal of the coiled ring within the pocket defined by the first part of the contact housing and prior to attachment of the second part to the first part.

### CROSS-REFERENCE TO RELATED APPLICATIONS

This application claims the benefit under 35 U.S.C. § 119 (e) of U.S. Provisional Patent Application Ser. No. 63/538,241, filed Sep. 13, 2024, which is incorporated herein by reference.

### FIELD

The present invention is directed to the area of implantable electrical stimulation systems and methods of making and using the systems. The present invention is also directed to implantable electrical stimulation systems having coil connectors, as well as methods of making and using the coiled ring contacts and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

At least one aspect is a connector assembly that includes a connector body having an elongated shape with a first end, an opposing second end, and a longitudinal axis, the connector body defining a port at the first end configured to receive a proximal part of a lead or lead extension; and a plurality of coiled ring contacts axially spaced-apart within the connector body and collectively forming a lumen that extends from the port along the longitudinal axis of the connector body. Each coiled ring contact includes a contact housing including a first part and a second part attached to the first part, wherein at least the first part includes a first radial sidewall and a first lateral sidewall extending laterally from the first radial sidewall, the first radial sidewall and the first lateral sidewall defining a pocket, and a coiled ring disposed within the contact housing, wherein the coiled ring contact is configured for disposal of the coiled ring within the pocket defined by the first part of the contact housing and prior to attachment of the second part to the first part, wherein the coiled ring is positioned within the contact housing so that insertion of the proximal part of the lead or lead extension through the coiled ring contact results in contact between the coiled ring and the lead or lead extension.

In at least some aspects, the coiled ring is disposed within the pocket defined by the first part of the contact housing without deformation of the coiled ring. In at least some aspects, the coiled ring is disposed within the pocket defined by the first part of the contact housing without asymmetric deformation of the coiled ring.

In at least some aspects, the second part includes a second radial sidewall and a second lateral sidewall extending laterally from the second radial sidewall. In at least some aspects, the first lateral sidewall and the second lateral sidewall are attached to each other by ends thereof. In at least some aspects, the first lateral sidewall is disposed radially inward from the second lateral sidewall. In at least some aspects, the first part further includes a perimeter sidewall extending laterally from the first lateral sidewall, wherein the perimeter sidewall has a radial thickness that is less than a radial thickness of the first lateral sidewall, wherein at least a portion of the second lateral sidewall is disposed radially inward from the perimeter sidewall.

In at least some aspects, the first part further includes a first lateral tab extending laterally from the first lateral sidewall, wherein the perimeter sidewall has a radial thickness that is less than a radial thickness of the first lateral sidewall, wherein at least a portion of the second part is disposed radially inward from the first lateral tab. In at least some aspects, the second part is a flat disc having an opening therethrough.

In at least some aspects, the first and second parts define a groove within which at least a portion of the coiled ring is disposed, wherein the groove has at least a portion with a circular, semicircular, elliptical, or oval shape. In at least some aspects, the first and second parts define a groove within which at least a portion of the coiled ring is disposed, wherein the groove has at least a portion with a triangular shape. In at least some aspects, the first and second parts define a groove within which at least a portion of the coiled ring is disposed, wherein the groove has at least a portion with a square or rectangular shape.

Another aspect is a lead assembly that includes a lead or a lead extension having a proximal portion and a distal portion, wherein the proximal portion of the lead or the lead extension includes a plurality of terminals electrically insulated from one another; and any of the connector assemblies described above.

A further aspect is an electrical stimulating system that includes the lead assembly described above; and a control module coupled to the lead assembly, the control module including a housing, and an electronic subassembly disposed in the housing, wherein connector assembly is part of the control module.

Yet another aspect is an electrical stimulating system that includes the lead assembly described above; and a lead extension coupleable to the control module and the lead, wherein the connector assembly is part of the lead extension.

Another aspect is a coiled ring contact that includes a contact housing including a first part and a second part attached to the first part, wherein at least the first part includes a first radial sidewall and a first lateral sidewall extending laterally from the first radial sidewall, the first radial sidewall and the first lateral sidewall defining a pocket; and a coiled ring disposed within the contact housing, wherein the coiled ring contact is configured for disposal of the coiled ring within the pocket defined by the first part of the contact housing and prior to attachment of the second part to the first part, wherein the coiled ring is positioned within the contact housing so that insertion of a proximal part of a lead or lead extension through the coiled ring contact results in contact between the coiled ring and the lead or lead extension. Any of the aspects regarding the coiled ring contact presented above can be applied to this coiled ring contact.

A further aspect is a method of making any of the coiled ring contacts described above. The method includes disposing the coiled ring into the pocket defined by the first radial sidewall and the first lateral sidewall of the first part; and attaching the second part to the first part with the coiled ring disposed therebetween.

In at least some aspects, the second part includes a second radial sidewall and a second lateral sidewall extending laterally from the second radial sidewall, wherein attaching the second part to the first part includes attaching the first lateral sidewall to the second lateral sidewall by ends thereof. In at least some aspects, the second part is a flat disc having an opening there though. In at least some aspects, the second part includes a second radial sidewall and a second lateral sidewall extending laterally from the second radial sidewall, wherein the first lateral sidewall is disposed radially inward from the second lateral sidewall.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic side view of one embodiment of a lead extension suitable for coupling with the electrical stimulation lead of FIG. 2

FIG. 4 is a schematic side view of one embodiment of the lead of FIG. 2 coupled to the lead extension of FIG. 3;

FIG. 5 is a schematic side view of one embodiment of a control module suitable for receiving either the lead of FIG. 2 or the lead extension of FIG. 3;

FIG. 6 is a schematic side view of one embodiment of an elongated member retained by the control module of FIG. 5;

FIG. 7A is a schematic cross-sectional view of one embodiment of coiled ring contact;

FIG. 7B is a schematic cross-sectional view of a portion of the coiled ring contact of FIG. 7A illustrating a coiled ring seated in a pocket defined by a first part of a contact housing;

FIG. 8 is a schematic cross-sectional view of another embodiment of coiled ring contact;

FIG. 9 is a schematic cross-sectional view of a third embodiment of coiled ring contact;

FIG. 10 is a schematic cross-sectional view of a fourth embodiment of coiled ring contact;

FIG. 11 is a schematic cross-sectional view of a fifth embodiment of coiled ring contact;

FIG. 12 is a schematic cross-sectional view of a sixth embodiment of coiled ring contact;

FIG. 13 is a schematic cross-sectional view of a seventh embodiment of coiled ring contact;

FIG. 14A is a schematic cross-sectional view of one embodiment of a portion of a coiled ring contact illustrating a shape of a groove;

FIG. 14B is a schematic cross-sectional view of one embodiment of a portion of a coiled ring contact illustrating another shape of the groove;

FIG. 14C is a schematic cross-sectional view of one embodiment of a portion of a coiled ring contact illustrating a third shape of the groove;

FIG. 15A is a schematic top view of an eighth embodiment of coiled ring contact prior to assembly;

FIG. 15B is a schematic top view of the coiled ring of contact of FIG. 15A after assembly;

FIG. 15C is a schematic cross-sectional view of the coiled ring contact of FIG. 15A; and

FIG. 16 is a schematic overview of one embodiment of components of an electrical stimulation system.

### DETAILED DESCRIPTION

The present invention is directed to the area of implantable electrical stimulation systems and methods of making and using the systems. The present invention is also directed to implantable electrical stimulation systems having coil connectors, as well as methods of making and using the coiled ring contacts and electrical stimulation systems.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

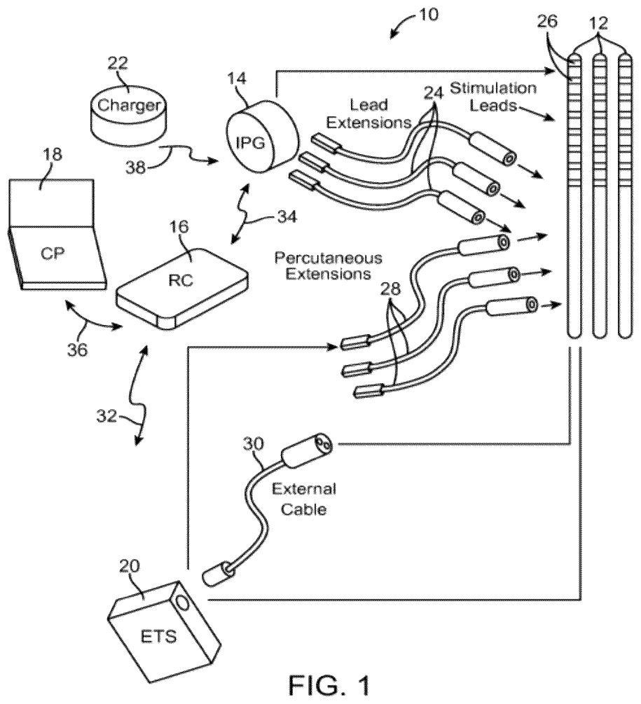

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

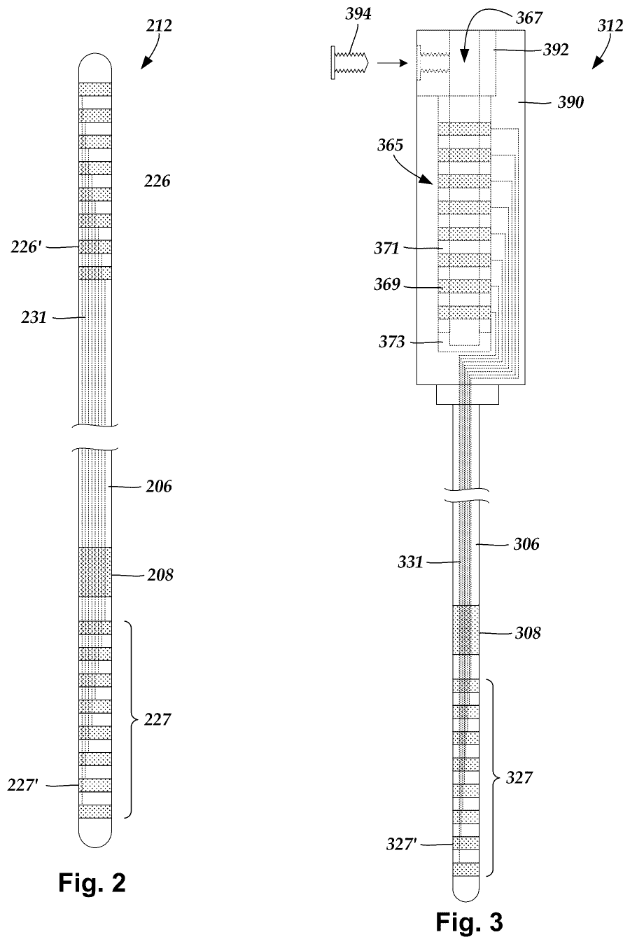

Turning to FIG. 2, one or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

Percutaneous leads are described herein for clarity of illustration. It will be understood that paddle leads and cuff leads can be used in lieu of, or in addition to, percutaneous leads. The leads described herein include 8 electrodes. It will be understood that the leads could include any suitable number of electrodes. The leads described herein exclusively include ring electrodes. It will be understood that the leads can include a distal-tip electrode, or one or more segmented electrodes in lieu of, or in addition to one or more ring electrodes. Additionally, the term “elongated member” used herein includes leads (e.g., percutaneous, paddle, cuff, or the like), as well as intermediary devices (e.g., lead extensions, adaptors, splitters, or the like).

FIG. 2 shows, in schematic side view, one embodiment of a lead 212 suitable for implanting into a patient and providing electrical stimulation. In some embodiments, the lead 212 is coupled directly to a control module. In other embodiments, the lead 212 is coupled to the control module via one or more intermediary devices. In the illustrated embodiment, an array of electrodes 226, which includes electrode 226′, is disposed along a distal portion of a lead body 206 lead and an array of lead terminals 227, which includes lead terminal 227′, is disposed along a proximal portion of the lead body. Lead conductors, such as lead conductor 231, extend along a longitudinal length of the lead and electrically couple the array of electrodes 226 to the array lead terminals 227.

Conductors can extend along the longitudinal length of the lead within one or more lumens defined in the lead. In other instances, the conductors may extend along the lead within the lead body itself. The lead 212 includes an retention sleeve 208 disposed along the proximal portion of the body to facilitate coupling of the proximal portion of the lead to a connector. The connector may be disposed along a control module. Alternatively, the retention sleeve 208 can be used to facilitate coupling of the proximal portion of the lead to a connector of an intermediary device, such as a lead extension which, in turn, is coupled to a connector of a control module.

FIG. 3 shows, in schematic side view, one embodiment of a lead extension 312 suitable for implanting into a patient and coupling a lead, such as the lead 212, to a control module. The lead extension 312 includes a lead-extension body 306 having a distal portion and a proximal portion. A lead-extension connector 390 is disposed along the distal portion of the lead-extension body 306 and an array of lead-extension terminals 327, such as lead-extension terminal 327′, are disposed along the proximal portion of the lead-extension body 306.

The lead-extension connector 390 contains a lead-extension connector stack 365 that defines a connector lumen 367 configured to receive the proximal portion of an elongated member (e.g., lead 212). The lead-extension connector stack 365 includes lead-extension connector contacts, such as lead-extension connector contact 369, arranged along the connector lumen 367 and configured to electrically couple with terminals of the elongated member (e.g., lead 212) when the proximal portion of the elongated member is received by the lead-extension connector 390. The connector contacts are electrically isolated from one another by electrically-nonconductive spacers, such as spacer 371. In at least some embodiments, the spacers provide at least a partial seal to reduce, or even eliminate, seepage of fluid into the connector from the environment external to the connector. The connector stack may also include an end stop 373 to promote alignment of the elongated-member terminals with the lead-extension connector contacts.

The lead-extension connector 390 further includes a retention assembly for facilitating retention of the proximal portion of the elongated member (e.g., lead 212) when the proximal portion of the elongated member is received by the lead-extension connector 390. In the illustrated embodiment, the retention assembly includes a lead-extension retention block 392. The lead-extension retention block 392 is positioned to align with the retention sleeve (208 in FIG. 2) of the elongated member when the elongated member is received by the lead-extension connector 390. In the illustrated embodiment, the retention assembly further includes a retaining member (e.g., a set screw, a pin, or the like) 394 for pressing the retention sleeve of the inserted elongated member against the retention block to retain inserted elongated member within the lead-extension connector 390.

Lead-extension conductors, such as lead-extension conductor 331, extend along a longitudinal length of the lead extension and electrically couple the lead-extension connector contacts to the array of lead-extension terminals 327. The lead-extension conductors can extend along the longitudinal length of the lead-extension body within one or more lumens defined in the lead extension. In other instances, the lead-extension conductors may extend along the lead extension within the lead-extension body itself. The lead extension 312 includes a retention sleeve 308 disposed along the proximal portion of the lead-extension body to facilitate coupling of the proximal portion of the lead extension to a connector, such as a control-module connector, another lead-extension connector, or the like.

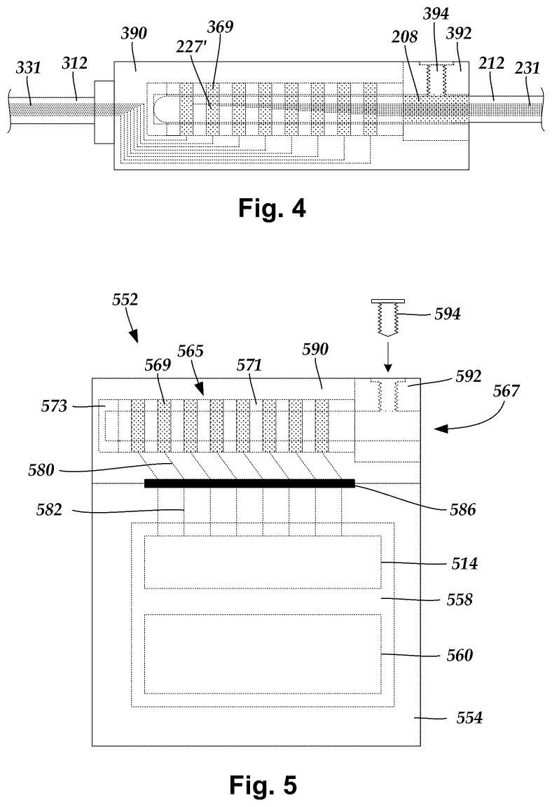

FIG. 4 shows, in schematic side view, one embodiment of the lead 212 received by the lead-extension connector 390. In the illustrated embodiment, the lead terminals 227, such as lead terminal 227′, are aligned with the lead-extension connector contacts, such as lead-extension connector contact 369. Accordingly, the lead conductors 231 are electrically coupled with the lead-extension conductors 331. Additionally, in the illustrated embodiment the lead retention sleeve 208 is aligned with the lead-extension retention block 392 and the retaining member 394 is pressing the lead retention sleeve 208 against the lead-extension retention block to retain the lead 212 within the lead-extension connector 390.

FIG. 5 shows, in schematic cross-sectional side view, one embodiment of a control module 552 suitable for coupling with an elongated member (e.g., the lead 212, the lead extension 312, or other intermediary device). The control module 552 includes a housing having a sealed portion 554 that houses an electronic subassembly 558 with a pulse generator 514 and, optionally, a power supply 560.

The housing further includes an unsealed portion that includes a connector 590 configured to receive an elongated device (e.g., the lead 212, the lead extension 312, or other intermediary device). Optionally, the connector 590 is positioned along an outer surface of the sealed portion of the housing. The connector 590 contains a connector stack 565 that defines a connector lumen 567 configured to receive the proximal portion of the elongated member. The connector stack 565 includes an array of connector contacts, such as connector contact 569, arranged along the connector lumen 567 and configured to electrically couple with terminals of the elongated member when the proximal portion of the elongated member is received by the connector 590. The connector contacts are electrically isolated from one another by electrically-nonconductive spacers, such as spacer 571. The connector stack may also include an end stop 573 to promote alignment of the elongated-member terminals with the connector contacts.

Feedthrough interconnects, such as feedthrough interconnect 582, are electrically coupled to the electrical subassembly 558 and extend within the sealed portion of the housing to a feedthrough interface 586 disposed along an interface between the sealed and unsealed portions of the housing. The connector contacts are electrically coupled to interconnect wires, such as interconnect wire 580, that extend along the unsealed portion of the housing and electrically couple the connector contacts to the feedthrough interconnects at the feedthrough interface 586. In some embodiments, the connector 590 is positioned along an outer surface of the sealed housing over the feedthrough interface 586. In other embodiments, the connector 590 is disposed at least partially within an outer surface of the sealed housing.

The connector 590 further includes a retention assembly for facilitating retention of the proximal portion of the elongated member when the proximal portion of the elongated member is received by the control module 552. In the illustrated embodiment, the retention assembly includes a retention block 592. The retention block 592 is positioned to align with the retention sleeve (208 in FIG. 2; 308 in FIG. 3) of the elongated member when the elongated member is received by the control module 552. In the illustrated embodiment, the retention assembly further includes a retaining member (e.g., a set screw, a pin, or the like) 594 for pressing the retention sleeve of the inserted elongated member against the retention block to retain inserted elongated member within the control module 552.

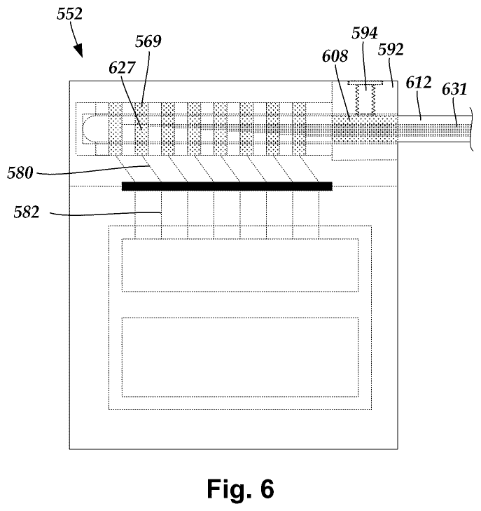

FIG. 6 shows, in schematic side view, one embodiment of an elongated member 612 (e.g., the lead 212, the lead extension 312, or other intermediary device) received by the control module 552. In the illustrated embodiment, the elongated-member terminals, such as elongated-member terminal 627, are aligned with the connector contacts, such as connector contact 569. Accordingly, the elongated-member conductors 631 are electrically coupled with the interconnect wires 580 and feedthrough interconnects 582. Additionally, in the illustrated embodiment a retention sleeve 608 disposed along the elongated member 612 is aligned with the retention block 592 and the retaining member 594 is pressing the elongated-member retention sleeve 608 against the retention block to retain the elongated member 612 within the control module 552.

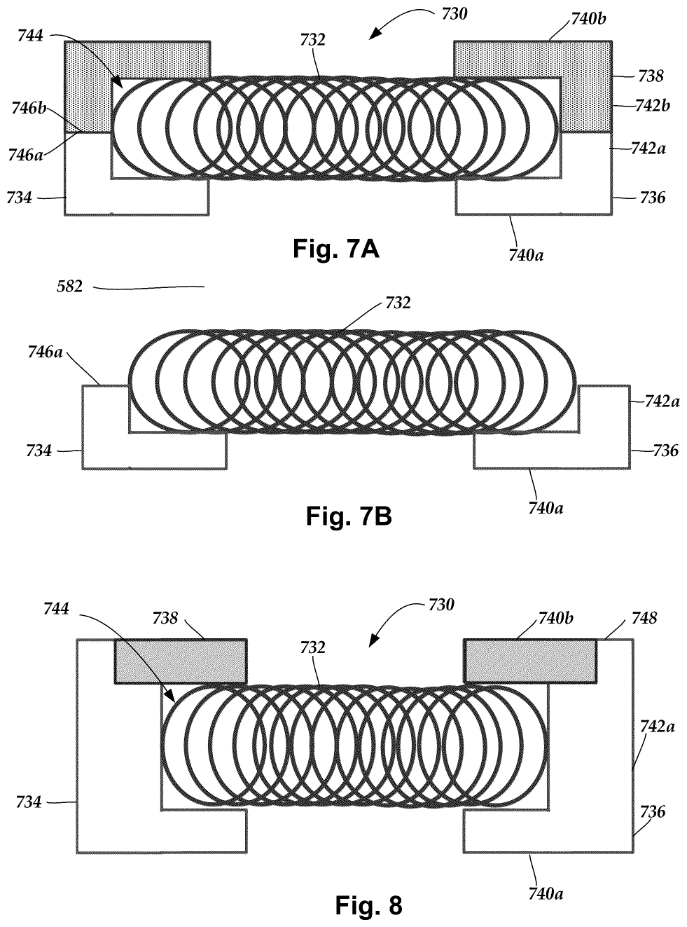

One example of a connector contact (e.g., connector contact 369 or 569) that can be used in a control module or a lead extension is a coiled ring contact. A coiled ring contact (e.g., canted coil contact) includes a conductive coiled ring (e.g., canted coil) disposed in a conductive contact housing. The coiled ring can be deformed or damaged during conventional methods for assembly of the coiled ring contact. In at least some embodiments, the coiled ring is a garter spring, which is a coiled spring that is connected at each end, or continuous, creating a ring shape. Examples of coiled ring contacts 730 are presented in FIGS. 7A to 13 and 15A to 15C. In many conventional coiled ring contacts, the contact housing is a single piece or formed prior to insertion of the coiled ring. The contact housing of these conventional coiled ring contacts includes a groove, pocket, or other arrangement that into which the coiled ring is inserted and includes sidewalls that retain the coiled ring within the contact housing. In order to insert the coiled ring into the groove of the pre-made housing, the coiled ring must be deformed (e.g., squeezed or pushed) to seat the coiled ring in the groove or pocket of the contact housing. Often, the deformation is asymmetric which can result in asymmetric deformation of the coiled ring. For example, a portion of the coiled ring can be placed in the groove or pocket and the remainder of the coiled ring is pushed over at least one of the sidewalls of the contact housing. The deformation required for insertion of the coiled ring may result in a permanent deformation or may damage the coiled ring.

In contrast to these conventional coiled ring contacts, a coiled ring contact can include a contact housing that can be manufactured in at least two parts. One of the parts forms a pocket into which the coiled ring can be placed and then the second part is attached to the first part to retain the coiled ring in the contact housing.

FIG. 7A illustrates a first embodiment of a coiled ring contact 730 with a coiled ring 732 and a contact housing 734 having a first part 736 and a second part 738 attached to the first part. The first part 736 includes a first radial sidewall 740a and first lateral sidewall 742a which form a pocket 744 for receiving the coiled ring 732 prior to attachment of the second part 738 to the first part 736, as illustrated in FIG. 7B. (In the illustrated embodiment of FIG. 7A, “radial” refers to a direction parallel to a line running from left to right of FIG. 7A and “lateral” refers to a direction parallel to a line running from top to bottom of FIG. 7A. The coiled ring 732 defines a plane that is parallel to the line running from left to right of FIG. 7A.) In at least some embodiments, the coiled ring 732 is inserted into the pocket 744 without any deformation (or only minor deformation) of the coiled ring 732. In at least some embodiments, the coiled ring 732 is inserted into the pocket 744 without any asymmetric deformation of the coiled ring 732. In at least some embodiments, the first radial sidewall 740a may provide some symmetric compression of the coiled ring 732 to ensure electrical contact with the first radial sidewall 740a.

In the embodiment of FIG. 7A, the second part 738 includes a second radial sidewall 740b and a second lateral sidewall 742b. In the embodiment of FIG. 7A, the end surfaces 746a, 746b of the first and second lateral sidewalls 742a, 742b are directly attached after the coiled ring contact 730 is inserted into the pocket 744 formed by the first part 736.

The coiled ring 732 and the contact housing 734 can be formed of any suitable material including, but not limited to, stainless steel, titanium, platinum, or the like or any combination thereof, such as stainless steel coated with titanium or platinum. The first and second parts 736, 738 can be formed by any suitable method including, but not limited to, stamping, machining, molding, or the like or any combination thereof. The first and second parts 736, 738 can be formed using the same method or different methods.

Any suitable method can be used to attach the second part 738 of the contact housing 734 to the first part 736 including, but not limited to, laser welding, resist welding, TIG (tungsten inert gas) welding, MIG (metal inert gas) welding, other welding techniques, or the like or any combination thereof. The attachment may include one or more spot attachments (e.g., spot welding), one or more seam attachments (e.g., seam welding), or the like or any combination thereof.

FIG. 8 illustrates another embodiment of a coiled ring contact 730 with a coiled ring 732 and a contact housing 734 having a first part 736 and a second part 738 attached to the first part. During manufacturing, the coiled ring 732 is inserted into the pocket 744 formed by the first part 736 and then the second part 738 is attached to the first part.

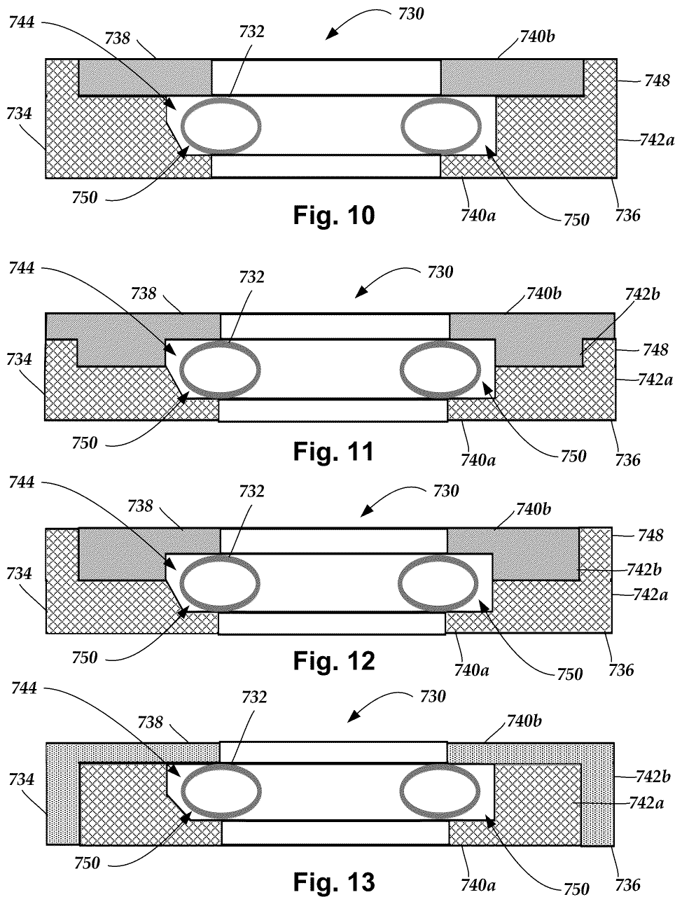

In addition to the first radial sidewall 740a and first lateral sidewall 742a, the first part 736 also includes a perimeter sidewall 748 extending from the first lateral sidewall 742a. The embodiments of FIGS. 10 (which is similar to the embodiment of FIGS. 8), 11, and 12 also include additional examples of the perimeter sidewall 748.

The radial thickness (e.g., the largest thickness in the radial direction) of the perimeter sidewall 748 is less than the radial thickness of the first lateral sidewall 742a. In at least some embodiments, the radial thickness of the perimeter sidewall 748 is no more than 75, 67, 60, 50, 40, 33, 25, or 20% of the thickness of the first lateral sidewall 742a.

The perimeter sidewall 748 surrounds at least a portion of the second part 738, as illustrated in FIGS. 8, 10, 11, and 12. That portion of the second part 738 fits within the perimeter sidewall 758 and is attached to at least the perimeter sidewall 748 and, optionally, the first lateral sidewall 742a.

In the embodiments of FIGS. 8 and 10, the second part 738 only includes a second radial sidewall 740b. In the embodiments of FIGS. 8 and 10, the second part 738 has the form of a disc with a central opening, which is circular in FIGS. 8a and 10 and can be oval or any other suitable shape.

In the embodiment of FIG. 11, the second part 738 includes a second radial sidewall 740b that extends over, and outside of, the perimeter sidewall 748, as well as a second lateral sidewall 742b that is surrounded by the perimeter sidewall 748. In at least some embodiments, the perimeter sidewall 748 can surround any selected portion of the second lateral sidewall 742b (for example, 10, 20, 25, 30, 33, 50, 66, 75, 90, or 100 percent of the second lateral sidewall). In at least some embodiments, the perimeter sidewall 748 can surround any selected portion of the second radial sidewall 740b (for example, 10, 20, 25, 30, 33, 50, 66, 75, 90, or 100 percent of the second radial sidewall). In the embodiment of FIG. 12, the perimeter sidewall 748 surrounds 100% of both the second lateral sidewall 742b and the second radial sidewall 740b. In the embodiments of FIGS. 8 and 10, the perimeter sidewall surrounds 100% of the second radial sidewall 740b and there is no second lateral sidewall.

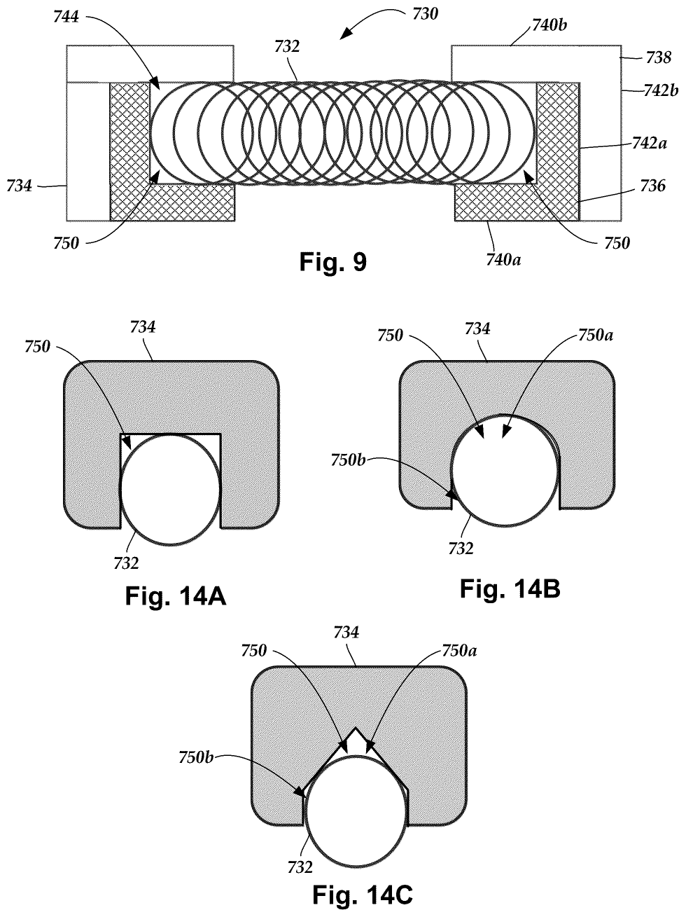

FIG. 9 illustrates another embodiment of a coiled ring contact 730 with a coiled ring 732 and a contact housing 734 having a first part 736 and a second part 738 attached to the first part. In this embodiment, the first lateral sidewall 740a is disposed radially inward from the second lateral sidewall 740b. In this embodiment, the second lateral sidewall 740a is also a perimeter sidewall 748. FIG. 13 illustrates another embodiment of this type of arrangement of a coiled ring contact 730.

The first and second parts 736, 738 of the contact housing 734 define a groove 750. FIGS. 14A, 14B, and 14C illustrate different shapes of the groove. In FIG. 14C, the groove 750 has a square or rectangular shape. The embodiments of FIGS. 7A, 8, and 9 also illustrate this shape and the embodiments of FIGS. 10, 11, 12, and 13 illustrate this shape on the right side. In contrast, the embodiments of FIGS. 10, 11, 12, and 13 illustrate a groove 750 that is asymmetrically shaped on the left side. It will be understood with respect to the embodiments of FIGS. 10, 11, 12, and 13 that the different groove shapes in these Figures are for illustrative purposes and that a contact housing 734 will typically have a uniform shape for the groove 750 (for example, either the square/rectangular shape on the right side of FIGS. 10, 11, 12, and 13 or the asymmetric shape on the left side of these Figures).

FIG. 14B illustrates a groove 750 having a circular, semicircular, oval, or elliptical shape for at least a portion 750a of the groove. FIG. 14C illustrates a groove 750 having a triangular shape for at least a portion 750a of the groove. In both FIGS. 14B and 14C, the groove 750 includes another portion 750b having a square/rectangular shape to facilitate fitting the circular/oval shape of the ringed coil 732.

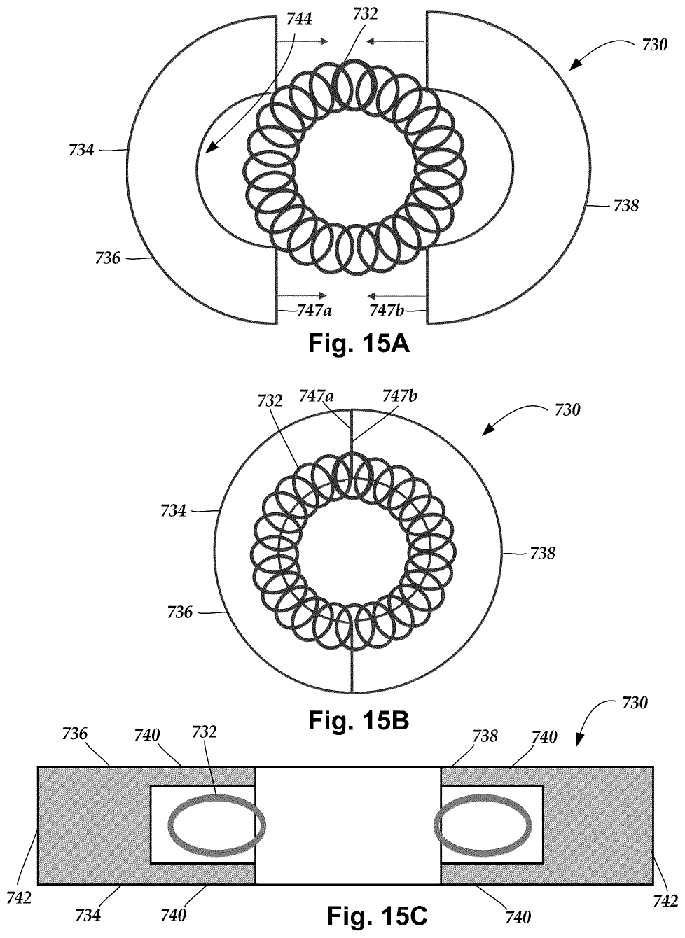

FIGS. 15A, 15B, and 15C illustrate a further embodiment of a coiled ring contact 730 with a coiled ring 732 and a contact housing 734 having a first part 736 and a second part 738 attached to the first part. In the embodiment of FIG. 7A, the first and second parts 736 and 738 are joined along surfaces 746a, 746b which define planes parallel to a plane defined by the coiled ring. In contrast, in the embodiment of FIGS. 15A, 15B, and 15C, the first and second parts are joined along surfaces 747a, 747b which define planes perpendicular to a plane defined by the coiled ring. The first and second parts 736, 738 both include two radial sidewalls 740 and a lateral sidewall 742. In the illustrated embodiment of FIGS. 15A, 15B, and 15C, the outer perimeter of the first and second parts 736, 738 form halves of a ring. It will be understood that the outer perimeter of the first and second parts 736, 738 can have other shapes including, but not limited to, two parts of an oval, two parts of a square or rectangle, two parts of a hexagon or octagon or other polygon, or the like.

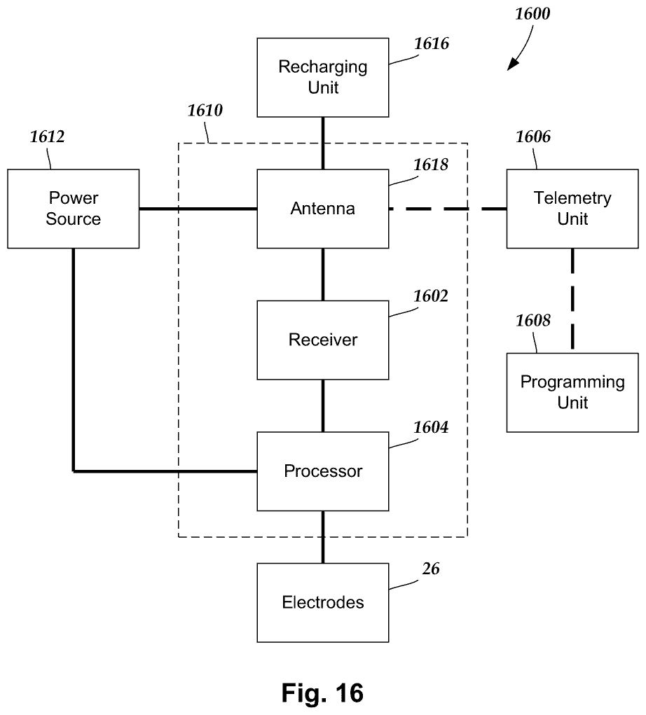

FIG. 16 is a schematic overview of one embodiment of components of an electrical stimulation system 1600 including an electronic subassembly 1610 disposed within a control module. It will be understood that the electrical stimulation system can include more, fewer, or different components and can have a variety of different configurations including those configurations disclosed in the stimulator references cited herein.

Some of the components (for example, a power source 1612, an antenna 1618, a receiver 1602, and a processor 1604) of the electrical stimulation system can be positioned on one or more circuit boards or similar carriers within a sealed housing of an implantable pulse generator, if desired. Any power source 1612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1618 or a secondary antenna. The external power source can be in a device that is mounted on the skin of the user or in a unit that is provided near the user on a permanent or periodic basis.

If the power source 1612 is a rechargeable battery, the battery may be recharged using the optional antenna 1618, if desired. Power can be provided to the battery for recharging by inductively coupling the battery through the antenna to a recharging unit 1616 external to the user. Examples of such arrangements can be found in the references identified above.

In one embodiment, electrical current is emitted by the electrodes 26 on the paddle or lead body to stimulate nerve fibers, muscle fibers, or other body tissues near the electrical stimulation system. The processor 1604 is generally included to control the timing and electrical characteristics of the electrical stimulation system. For example, the processor 1604 can, if desired, control one or more of the timing, frequency, strength, duration, and waveform of the pulses. In addition, the processor 1604 can select which electrodes can be used to provide stimulation, if desired. In some embodiments, the processor 1604 selects which electrode(s) are cathodes and which electrode(s) are anodes. In some embodiments, the processor 1604 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1608 that, for example, allows modification of pulse characteristics. In the illustrated embodiment, the processor 1604 is coupled to a receiver 1602 which, in turn, is coupled to the optional antenna 1618. This allows the processor 1604 to receive instructions from an external source to, for example, direct the pulse characteristics and the selection of electrodes, if desired.

In one embodiment, the antenna 1618 is capable of receiving signals (e.g., RF signals) from an external telemetry unit 1606 which is programmed by the programming unit 1608. The programming unit 1608 can be external to, or part of, the telemetry unit 1606. The telemetry unit 1606 can be a device that is worn on the skin of the user or can be carried by the user and can have a form similar to a pager, cellular phone, or remote control, if desired. As another alternative, the telemetry unit 1606 may not be worn or carried by the user but may only be available at a home station or at a clinician's office. The programming unit 1608 can be any unit that can provide information to the telemetry unit 1606 for transmission to the electrical stimulation system 1600. The programming unit 1608 can be part of the telemetry unit 1606 or can provide signals or information to the telemetry unit 1606 via a wireless or wired connection. One example of a suitable programming unit is a computer operated by the user or clinician to send signals to the telemetry unit 1606.

The signals sent to the processor 1604 via the antenna 1618 and the receiver 1602 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1600 to cease operation, to start operation, to start charging the battery, or to stop charging the battery. In other embodiments, the stimulation system does not include the antenna 1618 or receiver 1602 and the processor 1604 operates as programmed.

Optionally, the electrical stimulation system 1600 may include a transmitter (not shown) coupled to the processor 1604 and the antenna 1618 for transmitting signals back to the telemetry unit 1606 or another unit capable of receiving the signals. For example, the electrical stimulation system 1600 may transmit signals indicating whether the electrical stimulation system 1600 is operating properly or not or indicating when the battery needs to be charged or the level of charge remaining in the battery. The processor 804 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A connector assembly, comprising:
a connector body having an elongated shape with a first end, an opposing second end, and a longitudinal axis, the connector body defining a port at the first end configured to receive a proximal part of a lead or lead extension; and
a plurality of coiled ring contacts axially spaced-apart within the connector body and collectively forming a lumen that extends from the port along the longitudinal axis of the connector body, each coiled ring contact comprising
a contact housing comprising a first part and a second part attached to the first part, wherein at least the first part comprises a first radial sidewall and a first lateral sidewall extending laterally from the first radial sidewall, the first radial sidewall and the first lateral sidewall defining a pocket, and
a coiled ring disposed within the contact housing, wherein the coiled ring contact is configured for disposal of the coiled ring within the pocket defined by the first part of the contact housing and prior to attachment of the second part to the first part, wherein the coiled ring is positioned within the contact housing so that insertion of the proximal part of the lead or lead extension through the coiled ring contact results in contact between the coiled ring and the lead or lead extension.

2. The connector assembly of claim 1, wherein the coiled ring is disposed within the pocket defined by the first part of the contact housing without deformation of the coiled ring.

3. The connector assembly of claim 1, wherein the coiled ring is disposed within the pocket defined by the first part of the contact housing without asymmetric deformation of the coiled ring.

4. The connector assembly of claim 1, wherein the second part comprises a second radial sidewall and a second lateral sidewall extending laterally from the second radial sidewall.

5. The connector assembly of claim 4, wherein the first lateral sidewall and the second lateral sidewall are attached to each other by ends thereof.

6. The connector assembly of claim 4, wherein the first lateral sidewall is disposed radially inward from the second lateral sidewall.

7. The connector assembly of claim 4, wherein the first part further comprises a perimeter sidewall extending laterally from the first lateral sidewall, wherein the perimeter sidewall has a radial thickness that is less than a radial thickness of the first lateral sidewall, wherein at least a portion of the second lateral sidewall is disposed radially inward from the perimeter sidewall.

8. The connector assembly of claim 1, wherein the first part further comprises a first lateral tab extending laterally from the first lateral sidewall, wherein the perimeter sidewall has a radial thickness that is less than a radial thickness of the first lateral sidewall, wherein at least a portion of the second part is disposed radially inward from the first lateral tab.

9. The connector assembly of claim 1, wherein the second part is a flat disc having an opening therethrough.

10. The connector assembly of claim 1, wherein the first and second parts define a groove within which at least a portion of the coiled ring is disposed, wherein the groove has at least a portion with a circular, semicircular, elliptical, or oval shape.

11. The connector assembly of claim 1, wherein the first and second parts define a groove within which at least a portion of the coiled ring is disposed, wherein the groove has at least a portion with a triangular shape.

12. The connector assembly of claim 1, wherein the first and second parts define a groove within which at least a portion of the coiled ring is disposed, wherein the groove has at least a portion with a square or rectangular shape.

13. A lead assembly, comprising:
a lead or a lead extension having a proximal portion and a distal portion, wherein the proximal portion of the lead or the lead extension comprises a plurality of terminals electrically insulated from one another; and
the connector assembly of claim 1.

14. An electrical stimulating system comprising:
the lead assembly of claim 13; and
a control module coupled to the lead assembly, the control module comprising
a housing, and
an electronic subassembly disposed in the housing, wherein connector assembly is part of the control module.

15. An electrical stimulating system comprising:
the lead assembly of claim 13; and
a lead extension coupleable to the control module and the lead, wherein the connector assembly is part of the lead extension.

16. A coiled ring contact, comprising:
a contact housing comprising a first part and a second part attached to the first part, wherein at least the first part comprises a first radial sidewall and a first lateral sidewall extending laterally from the first radial sidewall, the first radial sidewall and the first lateral sidewall defining a pocket; and
a coiled ring disposed within the contact housing, wherein the coiled ring contact is configured for disposal of the coiled ring within the pocket defined by the first part of the contact housing and prior to attachment of the second part to the first part, wherein the coiled ring is positioned within the contact housing so that insertion of a proximal part of a lead or lead extension through the coiled ring contact results in contact between the coiled ring and the lead or lead extension.

17. A method of making the coiled ring contact of claim 16, the method comprising:
disposing the coiled ring into the pocket defined by the first radial sidewall and the first lateral sidewall of the first part; and
attaching the second part to the first part with the coiled ring disposed therebetween.

18. The method of claim 17, wherein the second part comprises a second radial sidewall and a second lateral sidewall extending laterally from the second radial sidewall, wherein attaching the second part to the first part comprises attaching the first lateral sidewall to the second lateral sidewall by ends thereof.

19. The method of claim 17, wherein the second part is a flat disc having an opening therethrough.

20. The method of claim 17, wherein the second part comprises a second radial sidewall and a second lateral sidewall extending laterally from the second radial sidewall, wherein the first lateral sidewall is disposed radially inward from the second lateral sidewall.
